(19) United States
(12) Patent Application Publication
(10) Pub. No.: US 2026/0027345 A1
(43) Pub. Date: Jan. 29, 2026

(54) APICAL CONNECTOR AND METHOD FOR IMPLANTING AND REMOVING A VENTRICULAR ASSIST DEVICE

(71) Applicant: BRIOHEALTH TECHNOLOGIES, INC., Jiangsu (CN)

(72) Inventors: Yunshan MA, Suzhou (CN); Sihan LIU, Suzhou (CN)

(73) Assignee: BRIOHEALTH TECHNOLOGIES, INC., Jiangsu (CN)

(21) Appl. No.: 19/119,697

(22) PCT Filed: Feb. 21, 2024
(86) PCT No.: PCT/CN2024/077913

§ 371 Date: Apr. 9, 2025

(30) Foreign Application Priority Data
Jan. 2, 2024 (CN) 202410002778.X

(51) Int. Cl. A61M 60/863; A61M 60/178

(52) U.S. Cl. CPC A61M 60/863; A61M 60/178; A61M 2205/0216

(57) ABSTRACT

The present disclosure relates an apical connector and a method for implanting and removing a ventricular assist device. The apical connector includes a first connection assembly and a second connection assembly. The first connection assembly includes a first base, a first positioning portion, and an annular cuff. The second connection assembly includes a second base and a first stopping portion. When the second base is mounted to the first through hole, the first stopping portion is located at a second position, and the first positioning portion is blocked on an end surface of the first stopping portion facing a first direction.

### TECHNICAL FIELD

The present disclosure relates to the technical field of medical devices, and in particular to an apical connector and a method for implanting and removing a ventricular assist device.

### BACKGROUND

The ventricular assist device, or blood pump, includes an inlet tube and an outlet tube. When implanted in the human body, the outlet tube is connected to an aorta through an artificial blood vessel. The inlet tube is inserted into a ventricle through an opening on an apex of a heart. In order to secure the ventricular assist device relative to the heart, the ventricular assist device is usually connected to the heart through an apical connector. The apical connector may include a cuff and a positioning member, the cuff is configured to be fixed to the heart through suturing, and the positioning member is connected to the inlet tube of the ventricular assist device, so that the ventricular assist device can be relatively fixed to the heart.

### SUMMARY

The present disclosure provides an apical connector and a method for implanting and removing a ventricular assist device.

An apical connector includes a first connection assembly and a second connection assembly. The first connection assembly includes a first base provided with a first through hole and a first positioning portion provided on an inner wall of the first through hole. The second connection assembly includes a second base and a first stopping portion. The second base is configured to be connected to a ventricular assist device. The second base is provided with a second through hole, and an inlet tube of the ventricular assist device is inserted into the second through hole. The first stopping portion is connected to the second base. When a part of the second base is inserted into the first through hole, the first stopping portion is elastically engaged with the first positioning portion.

In one of the embodiments, the first stopping portion has a first position at which the first stopping portion is elastically deformed radially towards an inner side of the second through hole and a second position at which the first stopping portion is elastically reset radially towards an outer side of the second through hole. When the part of the second base is inserted into the first through hole, the first stopping portion is located at the second position, the first positioning portion blocks on an end surface of the first stopping portion facing a first direction, and the first direction is opposite to an insertion direction of the second base.

In one of the embodiments, the second connection assembly further includes an elastic arm, the first stopping portion is connected to the elastic arm, the elastic arm is configured to be elastically deformed along a radial direction of the second through hole to drive the first stopping portion to be switched between the first position and the second position. The second base includes a first frame having an annular structure and provided with the second through hole on an inner side thereof; and a second frame sleeved on an outer side of the first frame in a circumferential direction thereof, and the second frame is provided with a first avoidance opening. A part of the elastic arm is located on a radial inner side of the second frame, and the first stopping portion protrudes from an outer surface of the second frame through the first avoidance opening.

In one of the embodiments, surfaces of the first frame and the second frame opposite to each other define a positioning cavity in communication with the first avoidance opening, and the elastic arm is accommodated in the positioning cavity.

In one of the embodiments, the first frame is provided with a second avoidance opening at a position corresponding to the first avoidance opening, and a size of the second avoidance opening along a circumferential direction of the second through hole is greater than a size of the first avoidance opening along a circumferential direction of the second through hole.

In one of the embodiments, the first frame is provided with a first anti-rotation groove, and the elastic arm includes a first anti-rotation portion engaged into the first anti-rotation groove.

In one of the embodiments, two first stopping portion are provided, a shape of the elastic arm is mated with a shape of an outer wall of the first frame, and the two first stopping portions are connected to two opposite ends of the elastic arm, respectively.

In one of the embodiments, the first frame is provided with a connection opening, the connection opening, the elastic arm, and the first stopping portion are provided in one-to-one correspondence. One end of each elastic arm is connected to an edge of the corresponding connection opening, and another end of each elastic arm is connected to the corresponding first stopping portion.

In one of the embodiments, the second frame is provided with a second anti-rotation groove, and the first frame is provided with a second anti-rotation portion engaged into the second anti-rotation groove. The second anti-rotation portion is provided at an edge of the connection opening, and a position of the second anti-rotation portion corresponds to a position of the first stopping portion in the circumferential direction of the second through hole.

In one of the embodiments, an end surface of the first stopping portion facing the second direction is an inclined surface, and the inclined surface is inclined towards the radially inner side of the second through hole.

In one of the embodiments, the first positioning portion includes a plurality of positioning segments spaced apart along a circumferential direction of the first through hole, and a plurality of first stopping portions are provided. When the second base is mounted into the first through hole, each of the positioning segments correspondingly blocks the end surface of the first stopping portion facing the first direction. A channel is formed between adjacent positioning segments forms a channel to allow the first stopping portion to extend through.

In one of the embodiments, the first connection assembly further includes a second positioning portion provided on the inner wall of the first through hole, and the second positioning portion includes a plurality of internal tooth structures. The first stopping portion is provided with an external tooth structure engaged with each of the internal tooth structures.

In one of the embodiments, the first connection assembly further includes an annular cuff, the cuff is connected to the first base and surrounds the first through hole. The first base includes a third frame having an annular structure, and the first through hole being formed on an inner side of the third frame; and a fourth frame sleeved on an outer periphery of the third frame, and the cuff being connected to the third frame.

In one of the embodiments, the first base further includes an annular abutting member connected to an end of the fourth frame facing a second direction and extends radially towards an outer side of the first through hole to an outer side of the fourth frame, the second direction is opposite to the first direction. At least a part of the cuff is supported by the abutting member.

In one of the embodiments, the end of the fourth frame facing the second direction is provided with a plurality of piercing portions spaced apart along the circumferential direction of the first through hole, the abutting member is provided with third through holes corresponding to the piercing portions, and each of the piercing portions extends through the corresponding third through hole to pierce the cuff.

In one of the embodiments, an end of the third frame facing the second direction is provided with a flange portion, and the cuff is sandwiched between the flange portion and the abutting member.

In one of the embodiments, the third frame includes an engaging block provided on an outer circumferential surface of the third frame, and inner peripheral sides of the abutting member and the fourth frame are provided with avoidance grooves. The fourth frame further provided with an engaging groove in communication with the avoidance groove on the fourth frame. The engaging block is engaged into engaging groove through the avoidance groove of the abutting member and the avoidance groove of the fourth frame.

In one of the embodiments, the fourth frame is provided with a plurality of fourth through holes, and the plurality of fourth through holes are spaced apart along the circumferential direction of the first through hole. The cuff is fixed to the fourth through hole through sutures.

In one of the embodiments, the first positioning portion is provided on an inner wall of the fourth frame and is located at an end of the fourth frame facing the first direction.

In one of the embodiments, the apical connector further includes a blocking member. The blocking member includes a blocking base and a plug. The blocking base includes an insertion portion inserted into the second through hole, the second connection assembly is provided outside the insertion portion. The plug is sleeved on the insertion portion and arranged sequentially with the second connection assembly along an axial direction of the second through hole.

In one of the embodiments, the plug includes a plug main body and a cover body covering the plug, and the cover body is provided with a plurality of pore structures.

A method for implanting and removing a ventricular assist device is provided, the ventricular assist device is implanted into or removed from a heart using the above-mentioned apical connector, the first connection assembly further includes an annular cuff connected to the first base. The method includes: sewing the cuff to a surface of the heart, and providing a hole on the heart at a position corresponding to the first through hole; providing the ventricular assist device connected to the second connection assembly; and driving, by an inlet pipe of the ventricular assist device, a part of the second base to extend through the first through hole and the hole until the first stopping portion is elastically engaged with the first positioning portion.

In one of the embodiments, prior to the sewing the cuff to the surface of the heart, the method further includes: connecting an end of a first blood vessel to an aorta of the heart; and connecting another end of the first blood vessel to an outlet tube of the ventricular assist device.

In one of the embodiments, the apical connector further includes a blocking member connected to the second connection assembly, and a part of the blocking member is inserted into the second through hole. The first positioning portion includes a plurality of positioning segments that are spaced apart along a circumferential direction of the first through hole, and a channel is formed between adjacent positioning segments to allow the first stopping portion to extend through. The method further includes: providing the blocking member connected to a second connection assembly; driving the ventricular assist device to drive the second connection assembly to rotate until the first stopping portion is aligned with the channel, and pulling out the ventricular assist device; and driving, by the blocking member, the second connection assembly to be inserted into the first through hole, and enabling the first stopping portion to cross the first positioning portion and be engaged with an end surface of the first positioning portion facing a second direction, the second direction being opposite to the first direction.

In one of the embodiments, prior to the driving the ventricular assist device to drive the second connection assembly to rotate until the first stopping portion is aligned with the channel and pulling out the ventricular assist device, the method further includes: disconnecting the first blood vessel from the aorta of the heart and blocking an opening of the aorta.

Details of one or more embodiments of the present disclosure are set forth in the following drawings and descriptions. These and other objects, purposes and advantages will become apparent upon review of the following specification, drawings and claims.

### BRIEF DESCRIPTION OF THE DRAWINGS

In order to illustrate the embodiments of the present disclosure more clearly, the drawings used in the embodiments will be described briefly. Apparently, the following described drawings are merely for the embodiments of the present disclosure, and other drawings can be derived by those of ordinary skill in the art without any creative effort.

FIG. 1 is a perspective view of an apical connector connected to a ventricular assist device according to an embodiment.

FIG. 2 is an exploded view of the apical connector of FIG. 1.

FIG. 3 is a partial exploded view of a second connection assembly of the apical connector of FIG. 2.

FIG. 4 is a perspective view of the apical connector connected to the ventricular assist device according to an embodiment.

FIG. 5 is an exploded view of the second connection assembly of the apical connector of FIG. 4.

FIG. 6 is an exploded view of an elastic arm of FIG. 5.

FIG. 7 is an exploded view of the second connection assembly of the apical connector according to another embodiment.

FIG. 8 is a perspective view of the second connection assembly of FIG. 7.

FIG. 9 is a cross-sectional view of the apical connector according to an embodiment.

FIG. 10 is a cross-sectional view of a first stopping portion of the apical connector being in contact with a first positioning portion according to an embodiment.

FIG. 11 is a partial cross-sectional view of the second connection assembly of the apical connector mounted to a first connection assembly according to an embodiment.

FIG. 12 is a perspective view of the first connection assembly of the apical connector according to an embodiment.

FIG. 13 is a perspective view of the first connection assembly of the apical connector according to an embodiment viewed from another aspect.

FIG. 14 is an exploded view of the first connection assembly of the apical connector according to an embodiment.

FIG. 15 is a cross-sectional view of the first connection assembly of the apical connector according to an embodiment.

FIG. 16 is an enlarged view of the first connection assembly of the apical connector according to an embodiment viewed from another aspect.

FIG. 17 is a perspective view of the first connection assembly of the apical connector according to another embodiment.

FIG. 18 is a cross-sectional view of the first connection assembly of the apical connector according to an embodiment.

FIG. 19 is a perspective view of the apical connector according to an embodiment viewed from another aspect.

FIG. 20 is a perspective view of a blocking member of the apical connector according to an embodiment.

FIG. 21 is a cross-sectional view of the blocking member of the apical connector according to an embodiment.

FIG. 22 is a perspective view of the blocking member of the apical connector mounted to the first connection assembly according to an embodiment.

FIG. 23 is a flowchart of a method for implanting and removing a ventricular assist device according to an embodiment.

Description of reference signs:

100. Apical connector;

10. First connection assembly; 11. First positioning portion; 111. Positioning segment; 1111. Channel; 12. First base; 121. First through hole; 1211. Chamfer structure; 123. Third frame; 1231, Engaging block; 1232, First counterbore hole; 1233, Second counterbore hole; 1234, Fastener; 124, Fourth frame; 1241, Piercing portion; 1242, Third through hole; 1243, Engaging groove; 1245. Fourth through hole; 1246. Suture hiding groove; 1247. First external tooth structure; 125. Flange portion; 1251. Pressing strip portion; 13. Cuff; 131. Identification portion; 14. Second positioning portion; 15. Abutting member; 151. First avoidance groove; 152. Second avoidance groove; 153. First weld; 154. Second weld;

20. Second connection assembly; 21. First stopping portion; 211. Inclined surface; 212. External tooth structure; 213. Second base; 221. Second through hole; 222. First frame; 2221. Second anti-rotation portion; 223. Second frame; 2230. Positioning boss; 2240. Positioning cavity; 2231. First avoidance opening; 2232. Second avoidance opening; 2233. Sealing portion; 2234. Second anti-rotation groove; 224. Blocking wall; 23. Elastic arm; 231. First anti-rotation groove; 232. First anti-rotation portion; 24. Sealing ring; 241. Mounting groove;

25. Connection opening;

30. Blocking member; 31. Blocking base; 310. Base body; 3101. Protrusion; 311. Insertion portion; 3111. Insertion hole; 32. Plug; 321. Plug main body; 322. Cover body; 323. Third weld; 324, Fourth weld;

200. Ventricular assist device; 210. Inlet tube; 2101. Inlet inner tube; 2102. Inlet outer tube; 2103. Flow channel; 220. Outlet tube; 230. Housing; 2300. Back cover; 240. Positioning groove; 250. Positioning notch; 260, Arrow;

400. Heart; 410. Aorta; 420. First blood vessel; 440. Cardiac apex;

F, First direction; S, Second direction.

### DETAILED DESCRIPTION OF THE EMBODIMENTS

In order to make the above objects, features and advantages of the present disclosure clear and easier to understand, the specific embodiments of the present disclosure are described in detail below in combination with the accompanying drawings. Many specific details are set forth in the following description to facilitate a full understanding of the present disclosure. However, the present disclosure can be implemented in many ways different from those described herein, and those skilled in the art can make similar improvements without departing from the connotation of the present disclosure. Therefore, the present disclosure is not limited by the specific embodiments disclosed below.

In the description of the present disclosure, it should be understood that the terms “center”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise”, “counterclockwise”, “axial”, “radial”, “circumferential direction” are based on the azimuth or position relationship shown in the attached drawings, which are only for the convenience of describing the present disclosure and simplifying the description, rather than indicating or implying that the device or element must have a specific azimuth, be constructed and operated in a specific azimuth, so such terms cannot be understood as a limitation of the present disclosure.

In addition, the terms “first” and “second” are only used for descriptive purposes and cannot be understood as indicating or implying relative importance or implicitly indicating the number of indicated technical features. Thus, the features defined with “first” and “second” may explicitly or implicitly include at least one of the features. In the description of the present disclosure, “a plurality of” means at least two, such as two, three, etc., unless otherwise expressly and specifically defined.

In the present invention, unless otherwise expressly specified and limited, the terms “mount”, “connect”, “contact”, “fix” and other terms should be understood in a broad sense, for example, they can be fixed connections, detachable connections, or integrated. They can be mechanical connection or electrical connection. They can be directly connected or indirectly connected through an intermediate medium. They can be the connection within two elements or the interaction relationship between two elements, unless otherwise expressly limited. For those skilled in the art, the specific meaning of the above terms in the present disclosure can be understood according to the specific situation.

In the present invention, unless otherwise expressly specified and limited, the first feature “above” or “below” the second feature may be in direct contact with the first and second features, or the first and second features may be in indirect contact through an intermediate medium. Moreover, the first feature is “above” the second feature, but the first feature is directly above or diagonally above the second feature, or it only means that the horizontal height of the first feature is higher than the second feature. The first feature is “below” of the second feature, which can mean that the first feature is directly below or obliquely below the second feature, or simply that the horizontal height of the first feature is less than that of the second feature.

It should be noted that when an element is called “fixed to” or “provided on” another element, it can be directly on another element or there can be a centered element. When an element is considered to be “connected” to another element, it can be directly connected to another element or there may be intermediate elements at the same time. The terms “vertical”, “horizontal”, “up”, “down”, “left”, “right” and similar expressions used herein are for the purpose of illustration only and do not represent the only embodiment.

The ventricular assist device, or blood pump, includes an inlet tube and an outlet tube. When implanted in the human body, the outlet tube is connected to an aorta through an artificial blood vessel. The inlet tube is inserted into a ventricle through an opening on an apex of a heart. In order to secure the ventricular assist device relative to the heart, the ventricular assist device is usually connected to the heart through an apical connector.

In the related art, the ventricular assist device includes a cuff and a fixing ring. The fixing ring includes a first half ring, a second half ring, and a threaded connecting member. The first half ring and the second half ring can enclose a clamping channel for insertion of the inlet tube of the ventricular assist device. One end of the first half ring is connected to one end of the second half ring are connected, and the other end of the first half ring is connected to the other of the second half ring through the threaded connecting member. The threaded connecting member can rotate about its own axis, so that the other end of the first half ring is adjacent to or away from the other end of the second half ring. A radial dimension of the clamping channel is adjusted in such a way, so that the clamping or loosening of the inlet tube can be adjusted.

However, the ventricular assist device is surgically implanted into a body. The implantation process of the ventricular assist device is complicated, time-consuming, and inconvenient, which increases the risk of operation.

According to an apical connector and a method for implanting and removing the ventricular assist device provided by the embodiments of the present disclosure, during the implantation process, the inlet tube connected to a second connection assembly is inserted into a first connection assembly to complete the mounting. It can be plug-and-play and is easy to operate, which can greatly save operation time and reduce the risk of operation.

An apical connector and a method for implanting and removing a ventricular assist device according to embodiments of the present disclosure will be described below with reference to the accompanying drawings.

A first aspect of the embodiment of the present disclosure provides an apical connector 100.

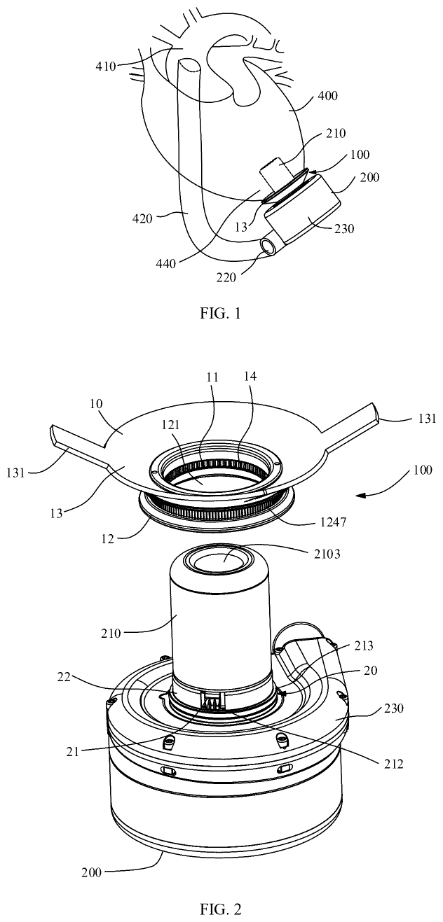

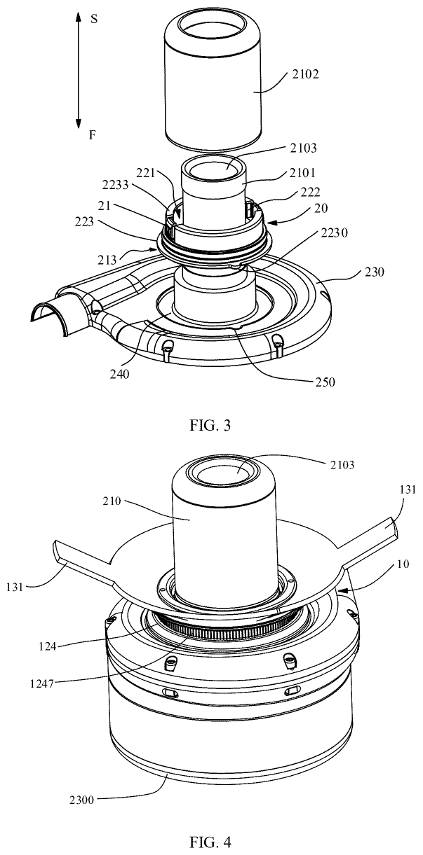

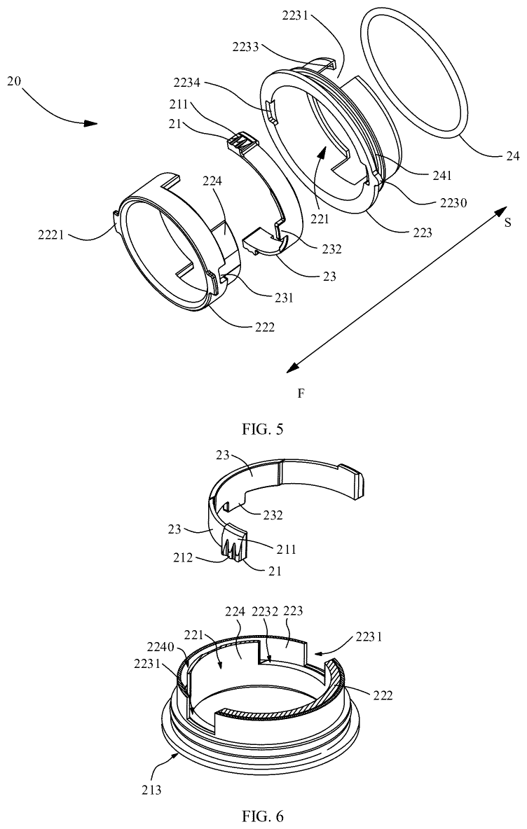

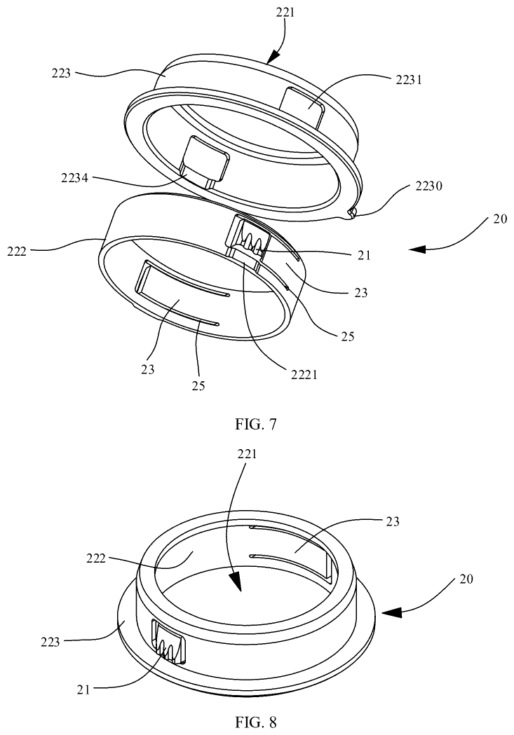

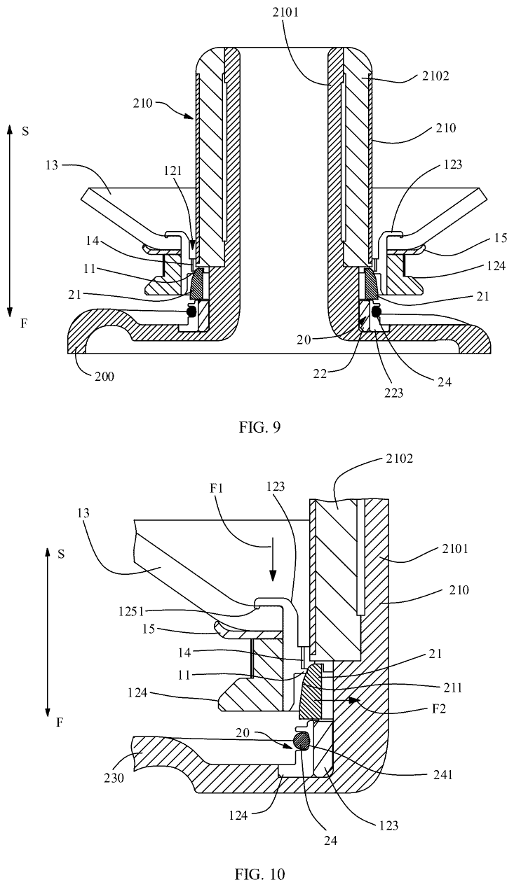

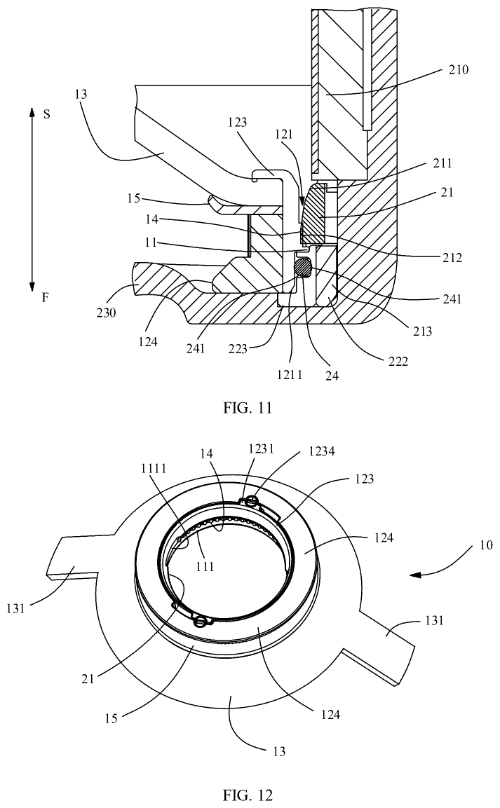

FIG. 1 is a perspective view of an apical connector 100 connected to a ventricular assist device 200 according to an embodiment. FIG. 2 is an exploded view of the apical connector 100 of FIG. 1. FIG. 3 is a partial exploded view of a second connection assembly 20 of the apical connector 100 of FIG. 2. FIG. 4 is a perspective view of the apical connector 100 connected to the ventricular assist device 200 according to an embodiment. FIG. 5 is an exploded view of the second connection assembly 20 of the apical connector 100 of FIG. 4. FIG. 6 is an exploded of an elastic arm 23 of FIG. 5. FIG. 7 is an exploded view of the second connection assembly 20 of the apical connector 100 according to another embodiment. FIG. 8 is a perspective view of the second connection assembly 20 of FIG. 7. FIG. 9 is a cross-sectional view of the apical connector 100 according to an embodiment. FIG. 10 is a cross-sectional view of a first stopping portion 21 of the apical connector 100 being in contact with a first positioning portion 11 according to an embodiment. FIG. 11 is a partial cross-sectional view of the second connection assembly 20 of the apical connector 100 mounted to the first connection assembly 10 according to an embodiment.

Referring to FIGS. 1, 4 and 9, the ventricular assist device 200 includes a housing 230, and the housing 230 is provided with an inlet tube 210 and an outlet tube 220, respectively. The inlet tube 210 includes an inner inlet tube 2101 and an outer inlet tube 2102 sleeved on the inner inlet tube 2101. The inlet inner tube 2101 and the inlet outer tube 2102 may be connected by welding. A welding position of the inlet inner tube 2101 and the inlet outer tube 2102 is located at an end of the inlet tube 210 in a direction of inserting into a heart 400. An inner side of the inlet inner tube 2101 defines a flow channel 2103 in communication with an interior of the ventricular assist device 200.

The connection between the heart 400 and the ventricular assist device 200 will be briefly described below.

FIG. 1 shows the ventricular assist device 200 implanted in the heart 400. One end of the first blood vessel 420 (for example, an artificial blood vessel or a natural blood vessel) is connected to an aorta 410 of the heart 400, and the other end of the first blood vessel 420 is connected to the outlet tube 220 of the ventricular assist device 200. A hole (not shown) is provided at a cardiac apex 440 of the heart 400, and the inlet tube 210 of the ventricular assist device 200 is inserted into a ventricle of the heart 400 through the hole.

Referring to FIGS. 2, 3, 4, 6 and 9, the apical connector 100 according to the embodiment of the present disclosure includes a first connection assembly 10 and a second connection assembly 20.

The first connection assembly 10 includes a first base 12 and a first positioning portion 11. The first base 12 is provided with a first through hole 121. The first positioning portion 11 is provided on an inner wall of the first through hole 121. Furthermore, the first connection assembly 10 further includes an annular cuff 13, which is connected to the first base 12 and surrounds the first through hole 121.

Referring to FIG. 6, the second connection assembly 20 includes a second base 213 and a first stopping portion 21. The second base 213 is configured to be connected to the ventricular assist device 200. The second base 213 is provided with a second through hole 221, and the second through hole 221 is configured to insert the inlet tube 210 of the ventricular assist device 200. The first stopping portion 21 is connected to the second base 213. The second connection assembly 20 is configured such that when a portion of the second base 213 is inserted into the first through hole 121, the first stopping portion 21 is elastically engaged with the first positioning portion 11.

With the above arrangement, during the implantation process, only the first connection assembly is connected to an organ to be implanted, and the inlet tube 210 connected to the second connection assembly 20 is inserted into the first connection assembly 10, so that the first stopping portion 21 is elastically engaged with the first positioning portion 11 to complete the mounting, which can realize plug-and-play and is easy to operate, which can greatly save operation time and reduce the risk of operation.

Further, the first stopping portion 21 has a first position at which the first stopping portion 21 is elastically deformed radially towards an inner side of the second through hole 221 and a second position at which the first stopping portion 21 is elastically reset radially towards an outer side of the second through hole 221.

Referring to FIG. 11, when the second base 213 is mounted into the first through hole 121, that is, when a part of the second base 213 is inserted into the first through hole 121. the first stopping portion 21 is located in the second position. The first positioning portion 11 blocks on an end surface of the first stopping portion 21 facing a first direction F. The first direction F is opposite to an insertion direction of the second base 213.

Referring to FIGS. 10 and 11, the first stopping portion 21 has a first position at which the first stopping portion 21 is elastically deformed radially towards an inner side of the second through hole 221 and a second position at which the first stopping portion 21 is elastically reset radially towards the outer side of the second through hole 221. As such, during the insertion of the inlet tube 210 of the ventricular assist device 200 into the first through hole 121 of the first connection assembly 10 by the second base 213, when the first stopping portion 21 is in contact with the first positioning portion 11, the first stopping portion 21 can be elastically deformed radially towards the inner side of the second through hole 221 under the pressure of a positioning portion 11, so that it is in the first position. After the first stopping portion 21 extends through the first positioning portion 11, the pressure of the first positioning portion 11 against the first stopping portion 21 is removed, and the first stopping portion 21 can elastically return to the second position radially toward the outer side of the second through hole 221. Since the first positioning portion 11 blocks the end surface of the first stopping portion 21 facing the first direction F, the second connecting component 20, that is, the ventricular assist device 200 can be prevented from withdrawing from the first through hole 121. In other words, during the implantation process, the mounting can be completed by inserting the inlet tube 210 connected to the second connection assembly 20 into the first connection assembly 10, which can achieve plug-and-play and is easy to operate, which can greatly save the operation time and reduce the risk of operation, and the overall size after connection is also small.

The second base 213 is configured to connect to the ventricular assist device 200. which means that the second base 213 can be fixed on the housing 230 of the main ventricular assist device. The second base 213 may be made of, for example, titanium alloy material. The ventricular assist device 200 may be, for example, a blood pump, and certainly, may also be other types of assist devices. The cuff 13 is made of flexible material, such as polyester material or polyethylene terephthalate (PET) material.

In the embodiment of the present disclosure, for convenience of explanation, a direction in which the second connection assembly 20 is pulled out of the first connection assembly 10 (i.e., a direction in which the ventricular assist device 200 moves away from the first connection assembly 10) is defined as the first direction F. A direction in which the second connection assembly 20 is inserted into the first connection assembly 10 (i.e., a direction in which the ventricular assist device 200 approaches the first connection assembly 10) is defined as a second direction S.

The specific structure of the second connection assembly 20 will be described below.

Referring FIGS. 3, 5 and 6, the second connection assembly 20 further includes an elastic arm 23. The first stopping portion 21 is connected to the elastic arm 23. The elastic arm 23 can be elastically deformed along a radial direction of the second through hole 221 to drive the first stopping portion 21 to be switched between the first position and the second position.

The second base 213 includes a first frame 222 and a second frame 223. The first frame 222 has an annular structure and provides with the second through hole 221 on an inner side of the first frame 222. The second frame 223 is sleeved on an outer side of the first frame 222 in a circumferential direction of the first frame 222. The second frame 223 is provided with a first avoidance opening 2231. A part of the elastic arm 23 is located on a radial inner side of the second frame 223, and the first stopping portion 21 protrudes from an outer surface of the second frame 223 through the first avoidance opening 2231.

In some embodiments, referring to FIGS. 3 and 6, surfaces of the first frame 222 and the second frame 223 opposite to each other define a positioning cavity 2240 in communication with the first avoidance opening 2231, and the elastic arm 23 is accommodated in the positioning cavity 2240. As a result, the first stopping portion 21 protrudes from the first avoidance opening 2231 to an outer side of the second frame 223 to facilitate the first stopping portion 21 to cooperate with the first positioning portion 11.

In order to prevent the elastic arm 23 from falling off the positioning cavity 2240, a sealing portion 2233 extending toward an inner side of the first frame 222 is also provided on the second frame 223. The sealing portion 2233 is annular and connected to an end of the second frame 223 facing the second direction S.

Further, the first frame 222 is provided with a second avoidance opening 2232 at a position corresponding to the first avoidance opening 2231, and a size of the second avoidance opening 2232 along a circumferential direction of the second through hole 221 is greater than a size of the first avoidance opening 2231 along a circumferential direction of the second through hole 221. In this way, when the elastic arm 23 moves radially inward, a space for the first stopping portion 21 to move inward is left.

Referring to FIGS. 5 and 6, further, the first frame 222 is provided with a first anti-rotation groove 231, and the elastic arm 23 includes a first anti-rotation portion 232 engaged into the first anti-rotation groove 231. In this way, the elastic arm 23 can be prevented from rotating relative to the first frame 222. For example, the first anti-rotation portion 232 is an anti-rotation plate extending from the elastic arm 23 in the first direction F. The first anti-rotation groove 231 is a shallow groove formed on an outer surface of the first frame 222.

In the embodiment of the present disclosure, two first stopping portions 21 may be provided. Certainly, it may also be another number according to actual needs. The situation when the number of the first stopping portions 21 is multiple is similar to this and will not be repeated herein.

When two first stopping portions 21 are provided, two second avoidance openings 2232 are also provided, and the two second avoidance openings 2232 are spaced apart along the circumferential direction of the second through hole 221. Certainly, a blocking wall 224 is provided between the two second avoidance openings 2232, and the elastic arm 23 can attached to and be supported on the outer surface (arc-shaped surface) of the blocking wall 224. In this way, the above-mentioned positioning cavity 2240 is defined between the blocking wall 224 and an inner wall of the second frame 223. During assembly, the elastic arm 23 and the first frame 222 are mounted into the second frame 223 together. Alternatively, in some other embodiments, the elastic arm 23 can be mounted into the second frame 223 from the first avoidance opening 2231 first, and then the first frame 222 can be assembled.

Further, a shape of the elastic arm 23 is mated with a shape of an outer wall of the first frame 222, and the two first stopping portions 21 are connected to two opposite ends of the elastic arm 23, respectively. For example, the elastic arm 23 may be configured in a semi-ring shape.

In the embodiment of the present disclosure, referring to FIG. 5, the second frame 223 is provided with a second anti-rotation groove 2234, and the first frame 222 is provided with a second anti-rotation portion 2221 engaged with the second anti-rotation groove 2234. In a specific embodiment, the second anti-rotation portion 2221 is a protruding structure provided at an end of the first frame 222 facing the first direction F. Two second anti-rotation portions 2221 may be provided, and the two second anti-rotation portions 2221 cooperate with the two second anti-rotation grooves 2234, respectively, so that the first frame 222 cannot rotate circumferentially relative to the second frame 223.

Referring to FIG. 3, FIG. 5 and FIG. 11, a mounting groove 241 is also provided on an outer surface of the second frame 223. A sealing ring 24 is provided in the mounting groove 241. The sealing ring 24 can protrude from the edge of the mounting groove 241, so as to interfere with the inner wall of the first through hole 121 of the first connection assembly 10 to act as a sealing and prevent blood in the ventricle from leaking. An opening edge of the first through hole 121 facing the first direction F is provided with a chamfer structure 1211. In this way, when the sealing ring 24 is mounted in the mounting groove 241 and the second connection assembly 20 is inserted into the first through hole 121, the sealing ring 24 protruding from the surface of the second frame 223 can be more easily mounted in the first through hole 121.

In the embodiment of the present disclosure, referring to FIGS. 10 and 11, an end surface of the first stopping portion 21 facing the second direction S is an inclined surface 211, and the inclined surface 211 is inclined towards the radially inner side of the second through hole 221. In this way, when the second connection assembly 20 is inserted into the first through hole 121 in the second direction S, the inclined surface 211 serves as a guide during the contact between the first stopper portion 21 and the first positioning portion 11.

Referring to FIGS. 7 and 8, the first frame 222 is provided with a connection opening 25. The numbers of the connection opening 25, the elastic arm 23, and the first stopping portion 21 are the same and the connection opening 25, the elastic arm 23, and the first stopping portion 21 are provided in one-to-one correspondence with each other. One end of each elastic arm 23 is connected to an edge of the corresponding connection opening 25, and the other end of each elastic arm 23 is connected to the corresponding first stopping portion 21. In this way, when the first stopping portion 21 is pressed, the elastic arm 23 is driven to move towards a radial inner side of the second through hole 221.

Further, the second frame 223 is provided with a second anti-rotation groove 2234, and the first frame 222 is provided with a second anti-rotation portion 2221 engaged into the second anti-rotation groove 2234, so as to prevent the first frame 222 and the second frame 223 from rotating in the circumferential direction. The second anti-rotation portion 2221 is provided at an edge of the connection opening 25, and a position of the second anti-rotation portion 2221 corresponds to a position of the first stopping portion 21 in the circumferential direction of the second through hole 221.

In this solution, the first frame 222 is integrally formed into a spring ring structure. After assembly, the first stopping portion 21 protrudes from the outside of the second frame 223 through the first avoidance opening 2231 provided on the second frame 223.

Referring to FIG. 2, in the embodiment of the present disclosure, the first connection assembly 10 further includes a second positioning portion 14. The second positioning portion 14 is provided on the inner wall of the first through hole 121, and the second positioning portion 14 includes a plurality of internal tooth structures. The first stopping portion 21 is provided with an external tooth structure 212 engaged with each of the internal tooth structures. In this way, when the second connection assembly 20 is mounted into the first through hole 121, the first stopping portion 21 is engaged with the second positioning portion 14 to prevent relative rotation between the second connection assembly 20 and the first connection assembly 10.

Further, referring to FIG. 4, a first external tooth structure 1247 is also distributed around an outer periphery of the fourth frame 124, and the first external tooth structure 1247 is configured to cooperate with a special surgical tool for circumferential clamping.

The cooperation between the second frame 223 and the housing 230 can be referred to FIG. 3 and FIG. 9. The housing 230 is provided with a positioning groove 240. An inner contour size of the positioning groove 240 is matched with that of an end of the second frame 223 facing the first direction F. In this way, the end of the second frame 223 facing the first direction F can be received in the positioning groove 240. In addition, the end of the second frame 223 facing the first direction F is provided with a positioning boss 2230, and the positioning groove 240 is provided with a positioning notch 250 at a position corresponding to the positioning boss 2230. In this way, the positioning boss 2230 can be engaged in the positioning notch 250 to achieve accurate positioning of the second frame 223 and the housing 230. In this case, the second frame 223 can be welded to the housing 230 by welding. Then, the outer inlet tube 2102 can be sleeved on the inner inlet tube 2101, and the ends of the outer inlet tube 2102 and the inner inlet tube 2101 in the second direction S can be welded.

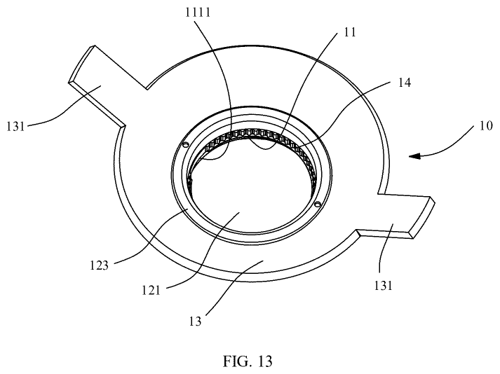

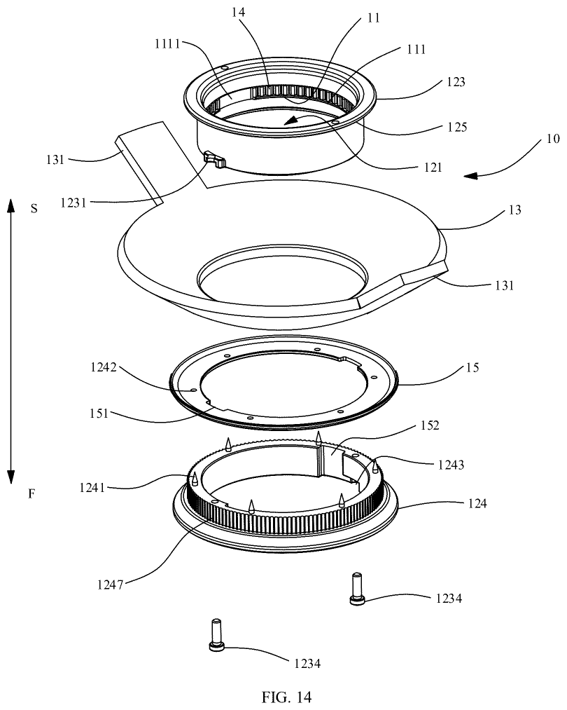

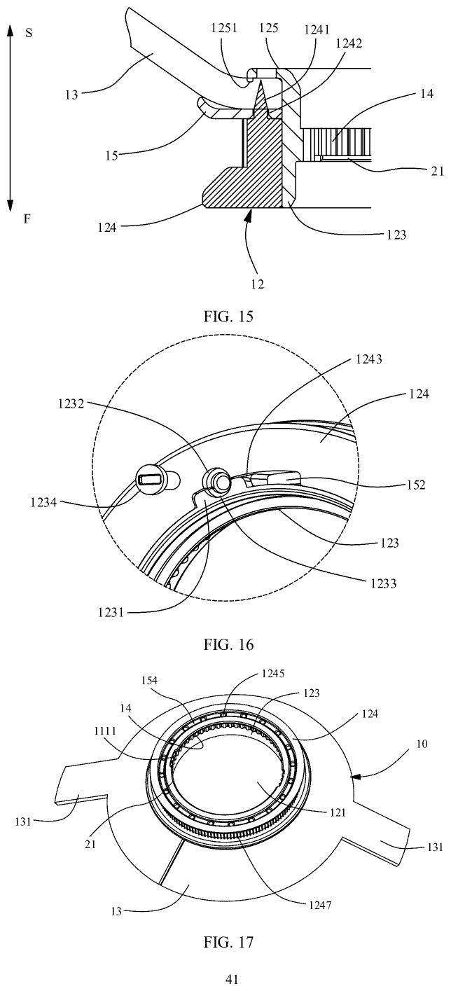

FIG. 12 is a perspective view of the first connection assembly 10 of the apical connector 100 according to an embodiment. FIG. 13 is a perspective view of the first connection assembly 10 of the apical connector 100 according to an embodiment viewed from another aspect. FIG. 14 is an exploded view of the first connection assembly 10 of the apical connector 100 according to an embodiment. FIG. 15 is a cross-sectional view of the first connection assembly 10 of the apical connector 100 according to an embodiment. FIG. 16 is an enlarged schematic view of the first connection assembly 10 of the apical connector 100 according to an embodiment viewed from another aspect.

Referring to FIGS. 11, 12, and 14, the first positioning portion 11 includes a plurality of positioning segments 111 spaced apart along a circumferential direction of the first through hole 121. A plurality of first stopping portions 21 are provided. When the second base 213 is mounted into the first through hole 121, each of the positioning segments 111 correspondingly blocks the end surface of the first stopping portion 21 facing the first direction F. A channel 1111 is formed between adjacent positioning segments 111 to allow the first stopping portion 21 to extend through. In this way, when the ventricular assist device 200 is removed, that is, when the second connection assembly 20 is pulled out relative to the first connection assembly 10, the second connection assembly 20 is rotated until the first stopping portion 21 is aligned with the channel 1111, then the second connection assembly 20 can be pulled out.

Referring to FIGS. 14 and 15, the first base 12 includes a third frame 123 and a fourth frame 124. The third frame 123 has an annular structure, and the first through hole 121 is formed on an inner side of the third frame 123. The fourth frame 124 is sleeved on an outer periphery of the third frame 123, and the cuff 13 is connected to the third frame 123.

Further, the first base 12 further includes an annular abutting member 15, which is connected to an end of the fourth frame 124 facing the second direction S, and extends radially towards an outer side of the first through hole 121 to an outer side of the fourth frame 124, and at least a part of the cuff 13 is supported by the abutting member 15. In this way, the cuff 13 can be better supported.

In the embodiment of the present disclosure, referring to FIGS. 14 and 15, the end of the fourth frame 124 facing the second direction S is provided with a plurality of piercing portions 1241 spaced apart along the circumferential direction of the first through hole 121. The abutting member 15 is provided with third through holes 1242 corresponding to the piercing portions 1241, and each of the piercing portions 1241 extends through the corresponding third through hole 1242 to pierce the cuff 13.

Furthermore, an end of the third frame 123 facing the second direction S is provided with a flange portion 125, and the cuff 13 is sandwiched between the flange portion 125 and the abutting member 15. In addition, in order to better press the cuff 13, a pressing strip portion 1251 is provided on an end surface of the flange portion 125 facing the first direction F. The pressing strip portion 1251 and the abutting member 15 cooperate with each other to better clamp the cuff 13. It should be understood that if a good sealing is not established between an apical opening and the inlet tube 210, blood will continue to flow out along a joint surface to an area of the apical connector 100. Through the above arrangement, a good sealing between the cuff 13 and the first base 12 can be achieved, thereby increasing the safety of the product.

Regarding the connection between the third frame 123 and the fourth frame 124, referring to FIGS. 14 and 16, the third frame 123 may include an engaging block 1231 provided on an outer peripheral surface thereof, inner peripheral sides of the abutting member 15 and the fourth frame 124 are provided with avoidance grooves. For example, a first avoidance groove 151 is provided on the abutting member 15, and a second avoidance groove 152 is provided on the fourth frame 124. The fourth frame 124 is further provided with an engaging groove 1243 in communication with the second avoidance groove 152 on the fourth frame 124. The engaging block 1231 is engaged with engaging groove 1243 through the first avoidance groove 151 of the abutting member 15 and the second avoidance groove 152 of the fourth frame 124. The engaging groove 1243 is configured to axially support the engaging block 1231 to prevent the third frame 123 from axially falling off.

A first counterbore hole 1232 is provided on an end surface of the fourth frame 124 facing the first direction F, and a second counterbore hole 1233 is provided on an end surface of the engaging block 1231 facing the first direction F. When the engaging block 1231 is engaged into the clamping groove 1243, the first counterbore hole 1232 and the second counterbore hole 1233 cooperatively form a complete counterbore hole. A fastener 1234 can be threadedly engaged into the complete counterbore hole, thereby preventing the fourth frame 124 and the engaging block 1231 from rotating relative to each other, that is, the fourth frame 124 and the third frame 123 are prevented from rotating relative to each other.

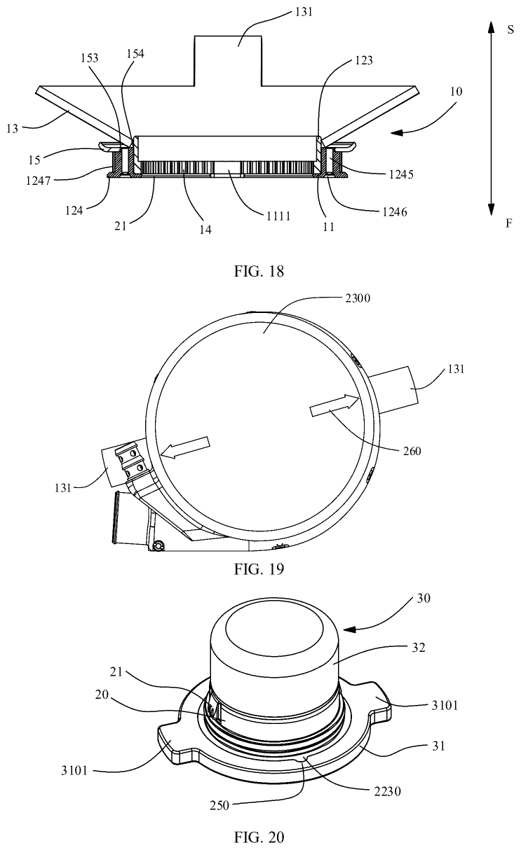

FIG. 17 is a perspective view of the first connection assembly 10 of the apical connector 100 according to another embodiment. FIG. 18 is a cross-sectional view of the first connection assembly 10 of the apical connector 100 according to an embodiment. FIG. 19 is a perspective view of the apical connector 100 according to an embodiment viewed from another aspect.

Referring to FIGS. 17 and 18, according to an alternative embodiment, the fourth frame 124 is provided with a plurality of fourth through holes 1245, and the plurality of fourth through holes 1245 are spaced apart along the circumferential direction of the first through hole 121, and the cuff 13 is fixed to the fourth through hole 1245 through sutures. The end of the fourth through hole 1245 in the first direction F is provided with a suture hiding hole.

Furthermore, the first positioning portion 11 is provided on an inner wall of the fourth frame 124 and is located at an end of the fourth frame 124 facing the first direction F.

In this embodiment, the abutting member 15 and the fourth frame 124 are fixed together by welding (such as laser welding). For example, the abutting member 15 is configured as a ring-shaped member, and the inner peripheral side of the abutting member 15 is fixed to the end surface of the fourth frame 124 facing the second direction S by laser welding. A first weld 153 is formed on the inner peripheral side of the abutting member 15.

The fourth frame 124 and the third frame 123 are also fixed by welding, and a second weld 154 is formed at a junction of the fourth frame 124 and the third frame 123. As mentioned above, the cuff 13 is fixed to the fourth frame 124 through sutures. In a specific embodiment, the sutures pass through each fourth through hole 1245 and are located in suture hiding grooves 1246, thereby ensuring that the sutures are not higher than the end surface of the fourth frame 124 facing the first direction F.

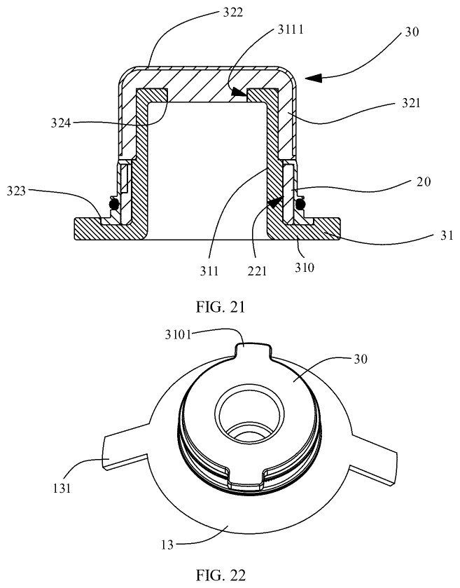

FIG. 20 is a perspective view of a blocking member 30 of the apical connector 100 according to an embodiment. FIG. 21 is a cross-sectional view of the blocking member 30 of the apical connector 100 according to an embodiment. FIG. 22 is a perspective view of the blocking member 30 of the apical connector 100 mounted to the first connection assembly 10 according to an embodiment.

Referring to FIGS. 20, 21 and 22, in the embodiment of the present disclosure, the apical connector 100 further includes a blocking member 30. The blocking member 30 includes a blocking base 31 and a plug 32.

The blocking base 31 includes a base body 310 and an insertion portion 311. The insertion portion 311 is connected to the second connection assembly 20, and the insertion portion 311 is inserted into the second through hole 221. The plug 32 is sleeved on the insertion portion 311 and is sequentially arranged with the second connection assembly 20 along an axial direction of the second through hole 221. The second frame 223 of the second connection assembly 20 can be connected to the base body 310 by welding, thus forming a third weld 323.

Further, the plug 32 includes a plug main body 321 and a cover body 322 covering outside of the plug 32. The cover body 322 is provided with a plurality of pore structures. The cover body 322 may be made by sintering or plasma spraying.

When the blocking member 30 is inserted into the first connection assembly 10, since the first stopping portion 21 is blocked by the first positioning portion 11, it will not come out of the first connection assembly 10. At this time, the plug 32 is inserted into the cardiac apex 440), and a myocardial tissue and an endothelial cell can grow on the pore structure of the cover body 322 to improve blood compatibility.

The plug 32 and the blocking base 31 can also be fixed by welding. For example, an inserting hole 3111 is provided on a top portion of the insertion portion 311, and a part of the plug main body 321 is engaged with the inserting hole 3111. A fourth weld 324 can be formed at a junction of the plug main body 321 and a wall of the inserting hole 3111.

Referring to FIGS. 13, 19, 20 and 21, a pair of protrusions 3101 are formed on the base body 310. The position of the pair of protrusions 3101 in the circumferential direction of the second through hole 221 corresponds to the first stopping portion 21. A pair of identification portions 131 are formed on the cuff 13. A position of the identification portions 131 in the circumferential direction of the first through hole 121 corresponds to a position of the channel 1111. When the blocking member 30 needs to be pulled out of the first connection assembly 10, the blocking member 30 only needs to be rotated relative to the first connection assembly 10 until the pair of first protrusions 3101 are respectively aligned with the pair of identification portions 131, which means that in the circumferential direction of the first through hole 121, the channel 1111 is aligned with first stopping portion 21, and the blocking member 30 can be pulled out at this time. As shown in FIG. 22, when the blocking member 30 is mounted on the first connection assembly 10, the pair of identification portions 131 and the pair of protrusions 3101 are circumferentially staggered from each other.

Referring to FIGS. 10 and 11, when the inlet tube 210 with the second connection assembly 20 is inserted into the first through hole 121 of the first connection assembly 10, the first positioning portion 11 will be in contact with the inclined surface 211 of the first stopping portion 21. When the inlet tube 210 is further inserted, the second connection assembly 20 is subjected to a force F1. This force F1 generates a component force F2 on the inclined surface 211, so that the first stopping portion 21 drives the elastic arm 23 to be elastically deformed, and the first stopping portion 21 is displaced inward and is located at the first position. When the inlet tube 210 is continuously pushed to enable the first stopping portion 21 to cross the first positioning portion 11, the force F2 exerted on the first stopping portion 21 is removed, and the first stopping portion 21 suddenly pops outwardly under an elastic force of the elastic arm 23, and hits the internal tooth structure of the second positioning portion 14 of the third frame 123, making a loud noise.

At this time, an end surface of the first stopping portion 21 facing the first direction F is axially blocked by the first positioning portion 11, so that the ventricular assist device 200 and the apical connector 100 are connected together without being axially disconnected. Thus, the purpose of the present disclosure is achieved. The sealing ring 24 is located in the mounting groove 241 of the fourth frame 124 and is in interference fit with an inner wall of the third frame 123 to establish a sealing between the ventricular assist device 200 and the apical connector 100, so that blood cannot flow out of the blood pump from the periphery of the inlet tube 210.

Referring to FIG. 2, the second positioning portion 14 is provided with an internal tooth structure, and the first stopping portion 21 is provided with an external tooth structure 212. The two are engaged and cooperate to prevent the ventricular assist device 200 from rotating axially relative to the apical connector 100. If an angle of the ventricular assist device 200 relative to the apical connector 100 needs to be adjusted, a special surgical tool can be used to clamp the first external tooth structure 1247 on the outer periphery of the fourth frame 124, and the ventricular assist device 200 is held with hand to apply a torque along an axis. At this time, the engaged second positioning portion 14 pushes the external tooth structure 212 on the first stopping portion 21, so that the elastic arm 23 is elastically deformed, and the first stopping portion 21 retracts inward, thereby achieving relative rotation.

After the ventricular assist device 200 is implanted into the heart 400, it is usually necessary to rotate the ventricular assist device 200 to adjust the angle to make the blood flow in the first blood vessel 420 smoother. In addition, during the operation, a doctor may need to insert and remove the ventricular assist device 200 multiple times. According to the apical connector 100 of the present disclosure, the ventricular assist device 200 can be freely rotated to adjust the angle, and is easy to pull out quickly, which is convenient for the doctor to operate and saving operation time.

Referring to FIGS. 14 and 19, if it is necessary to remove the ventricular assist device 200 from the apical connector 100, special surgical tools can be used to clamp the apical connector 100, and the ventricular assist device 200 can be rotated by hand until two arrows 260 on a blood pump back cover 2300 are aligned with the two identification portions 131 on the cuff 13. At this time, the first stopping portion 21 moves to the channel 1111, and can be removed by pulling it out with force.

In addition, it should be noted that referring to FIG. 11, after the second connection assembly 20 is inserted into the first through hole 121, a part of the structure of the second frame 223 is blocked by the fourth frame 124, so that it will not continue to move towards the heart 400 in the second direction S.

Certainly, the edge of the mounting groove 241 can also be blocked by the first positioning portion 11, so that the second frame 223 will not continue to move toward the heart 400 in the second direction S.

Alternatively, the housing 230 may be pressed against the fourth frame 124 to restrict a movement of the second frame 223 toward the heart 400 in the second direction S.

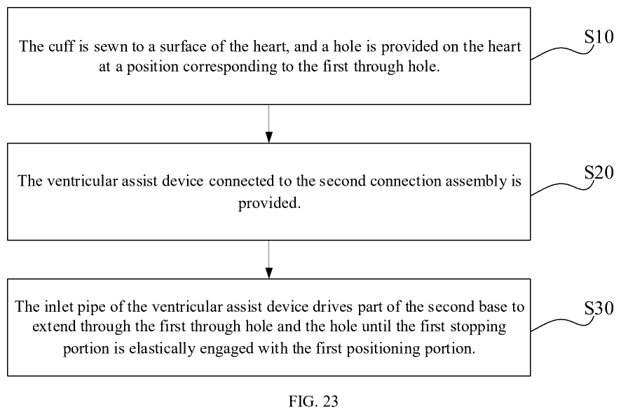

FIG. 23 is a flowchart of a method for implanting and removing a ventricular assist device 200 according to an embodiment of the present disclosure.

Referring to FIGS. 1 and 23, a second aspect of an embodiment of the present disclosure provides a method for implanting and removing the ventricular assist device 200. The ventricular assist device 200 is implanted into or removed from the heart 400 using the apical connector 100 in the previous embodiment. The structure, function, working principle, etc. of the ventricular assist device 200 have been described in detail previously and will not be described again herein.

The method for implanting and removing the ventricular assist device 200 provided by the embodiment of the present disclosure includes:

S10. The cuff is sewn to a surface of the heart, and a hole is provided on the heart at a position corresponding to the first through hole.

S20. The ventricular assist device connected to the second connection assembly is provided.

S30. The inlet pipe of the ventricular assist device drives part of the second base to extend through the first through hole and the hole until the first stopping portion is elastically engaged with the first positioning portion.

Further, the step of the inlet pipe of the ventricular assist device driving part of the second base to extend through the first through hole and the hole until the first stopping portion is elastically engaged with the first positioning portion specifically includes:

The inlet tube of the ventricular assist device drives a part of the second base to extend through the first through hole and the hole, so that the first stopping portion crosses the first positioning portion and is engaged with the end surface of the first positioning portion facing the second direction. The second direction is opposite to the first direction.

Further, referring to FIG. 1, in step S10, prior to sewing the cuff 13 to the surface of the heart 400, the method further includes:

One end of the first blood vessel 420 is connected to the aorta 410 of the heart 400, and the other end of the first blood vessel 420 is connected to the outlet tube 220 of the ventricular assist device 200.

Furthermore, as mentioned above, the apical connector 100 further includes a blocking member 30 connected to a second connection assembly 20, and a part of the blocking member 30 is inserted into the second through hole 221.

The first positioning portion 11 includes a plurality of positioning segments 111 that are spaced apart along the circumferential direction of the first through hole 121. A channel 1111 is formed between adjacent positioning segments 111 to allow the first stopping portion 21 to extend through.

The method for implanting and removing the ventricular assist device 200 further includes:

The ventricular assist device 200 is driven to drive the second connection assembly 20 to rotate until the first stopping portion 21 is aligned with the channel 1111, and the ventricular assist device 200 is pulled out.

A blocking member 30 connected to a second connection assembly 20 is provided.

The blocking member 30 drives the second connection assembly 20 to be inserted into the first through hole 121, and the first stopping portion 21 crosses the first positioning portion 11 and is engaged with the end surface of the first positioning portion 11 facing the second direction S. The second direction S is opposite to the first direction F.

In the embodiment of the present disclosure, prior to the step of rotating the ventricular assist device 200 until the first stopping portion 21 is aligned with the channel 1111 and pulling out the ventricular assist device 200, the method further includes:

The first blood vessel 420 is disconnected from the aorta 410 of the heart 400, and an opening of the aorta 410 is blocked.

A specific example is given below to illustrate a method for implanting and removing the apical connector 100 according to an embodiment of the present disclosure.

The implantation method includes:

Step 1: one end of the first blood vessel 420 is connected to the aorta 410 of the heart 400, and the other end of the first blood vessel 420 is connected to the outlet tube 220 of the ventricular assist device 200.

Step 2: the cuff 13 is sewn to the cardiac apex 440, and then a hole is provided on the cardiac apex 440 along the first through hole 121 of the first connection assembly 10.

Step 3: the ventricular assist device connected to the second connection assembly 20 is provided.

Step 4: the inlet tube 210 of the ventricular assist device 200 extends through the first through hole 121 and the hole, and is inserted toward the ventricle, so that the first stopping portion 21 crosses the first positioning portion 11 (a sound will be generated at the moment of crossing, indicating that the assembly is in place), and is engaged with the end surface of the first positioning portion 11 facing the second direction S. As shown in FIG. 4, at this time, the connection between the ventricular assist device 200 and the apical connector 100 is completed, that is, the connection between the ventricular assist device 200 and the heart 400 is completed.

After the patient's native heart 400 regains its function, the ventricular assist device needs to be removed. The removing method includes:

Step 1: the first blood vessel 420 is disconnected from the aorta 410 of the heart 400 and the opening of the aorta 410 is blocked.

Step 2: a blocking member 30 provided with the second connection assembly 20 is provided.

Step 3: the ventricular assist device 200 is driven to drive the second connection assembly 20 to rotate until the first stopping portion 21 is aligned with the channel 1111, and the ventricular assist device 200 is pulled out.

Step 4: the blocking member 30 drives the second connection assembly 20 to be inserted into the first through hole 121, and the first stopping portion 21 crosses the first positioning portion 11 and is engaged with the end surface of the first positioning portion 11 facing the second direction S.

The above-mentioned embodiments do not constitute a limitation on the protection scope of the technical solution. Any modifications, equivalent replacements and improvements made within the spirit and principles of the above-mentioned embodiments shall be included within the protection scope of this technical solution.

The foregoing descriptions are merely specific embodiments of the present disclosure, but are not intended to limit the protection scope of the present disclosure. Any variation or replacement readily figured out by a person skilled in the art within the technical scope disclosed in the present disclosure shall all fall within the protection scope of the present disclosure.

1. An apical connector, comprising:
a first connection assembly comprising:
a first base provided with a first through hole; and
a first positioning portion provided on an inner wall of the first through hole;
a second connection assembly comprising:
a second base configured to be connected to a ventricular assist device, the second base being provided with a second through hole, an inlet tube of the ventricular assist device being inserted into the second through hole; and
a first stopping portion connected to the second base;
wherein when a part of the second base is inserted into the first through hole, the first stopping portion is elastically engaged with the first positioning portion.

2. The apical connector according to claim 1, wherein the first stopping portion has a first position at which the first stopping portion is elastically deformed radially towards an inner side of the second through hole and a second position at which the first stopping portion is elastically reset radially towards an outer side of the second through hole;
when the part of the second base is inserted into the first through hole, the first stopping portion is located at the second position, the first positioning portion blocks an end surface of the first stopping portion facing a first direction, and the first direction is opposite to an insertion direction of the second base.

3. The apical connector according to claim 2, wherein the second connection assembly further comprises an elastic arm, the first stopping portion is connected to the elastic arm, the elastic arm is configured to be elastically deformed along a radial direction of the second through hole to drive the first stopping portion to be switched between the first position and the second position;
the second base comprises:
a first frame having an annular structure and provided with the second through hole on an inner side thereof, and
a second frame sleeved on an outer side of the first frame in a circumferential direction thereof, and the second frame is provided with a first avoidance opening;
wherein a part of the elastic arm is located on a radial inner side of the second frame, and the first stopping portion protrudes from an outer surface of the second frame through the first avoidance opening.

4. The apical connector according to claim 3, wherein surfaces of the first frame and the second frame opposite to each other define a positioning cavity in communication with the first avoidance opening, and the elastic arm is accommodated in the positioning cavity.

5. The apical connector according to claim 4, wherein the first frame is provided with a second avoidance opening at a position corresponding to the first avoidance opening, and a size of the second avoidance opening along a circumferential direction of the second through hole is greater than a size of the first avoidance opening along a circumferential direction of the second through hole.

6. The apical connector according to claim 4, wherein the first frame is provided with a first anti-rotation groove, and the elastic arm comprises a first anti-rotation portion engaged into the first anti-rotation groove.

7. The apical connector according to claim 6, wherein two first stopping portion are provided, a shape of the elastic arm is mated with a shape of an outer wall of the first frame, and the two first stopping portions are connected to two opposite ends of the elastic arm, respectively.

8. The apical connector according to claim 3, wherein the first frame is provided with a connection opening, the connection opening, the elastic arm, and the first stopping portion are provided in one-to-one correspondence, one end of each elastic arm is connected to an edge of the corresponding connection opening, and another end of each elastic arm is connected to the corresponding first stopping portion.

9. The apical connector according to claim 3, wherein the second frame is provided with a second anti-rotation groove, and the first frame is provided with a second anti-rotation portion engaged into the second anti-rotation groove, the second anti-rotation portion is provided at an edge of the connection opening, and a position of the second anti-rotation portion corresponds to a position of the first stopping portion in the circumferential direction of the second through hole.

10. The apical connector according to claim 3, wherein an end surface of the first stopping portion facing the second direction is an inclined surface, and the inclined surface is inclined towards the radially inner side of the second through hole.

11. The apical connector according to claim 3, wherein the first positioning portion comprises a plurality of positioning segments spaced apart along a circumferential direction of the first through hole, and a plurality of first stopping portions are provided;
when the second base is mounted into the first through hole, each of the positioning segments correspondingly blocks the end surface of the first stopping portion facing the first direction;
a channel is formed between adjacent positioning segments to allow the first stopping portion to extend through.

12. The apical connector according to claim 3, wherein the first connection assembly further comprises a second positioning portion provided on the inner wall of the first through hole, the second positioning portion comprises a plurality of internal tooth structures, and the first stopping portion is provided with an external tooth structure engaged with each of the internal tooth structures.

13. The apical connector according to claim 3, wherein the first connection assembly further comprises an annular cuff, the cuff is connected to the first base and surrounds the first through hole;
the first base comprises:
a third frame having an annular structure, and the first through hole being formed on an inner side of the third frame; and
a fourth frame sleeved on an outer periphery of the third frame, and the cuff being connected to the third frame.

14. The apical connector according to claim 13, wherein the first base further comprises an annular abutting member connected to an end of the fourth frame facing a second direction, the abutting member extends towards a radial outer side of the first through hole to an outer side of the fourth frame, the second direction is opposite to the first direction, and at least a part of the cuff is supported by the abutting member.

15. The apical connector according to claim 14, wherein the end of the fourth frame facing the second direction is provided with a plurality of piercing portions spaced apart along the circumferential direction of the first through hole, the abutting member is provided with third through holes corresponding to the piercing portions, and each of the piercing portions extends through the corresponding third through hole to pierce the cuff.

16. The apical connector according to claim 15, wherein an end of the third frame facing the second direction is provided with a flange portion, and the cuff is sandwiched between the flange portion and the abutting member.

17. The apical connector according to claim 14, wherein the third frame comprises an engaging block provided on an outer circumferential surface of the third frame, inner peripheral sides of the abutting member and the fourth frame are provided with avoidance grooves;
the fourth frame is further provided with an engaging groove in communication with the avoidance groove on the fourth frame;
the engaging block is engaged into engaging groove through the avoidance groove of the abutting member and the avoidance groove of the fourth frame.

18. The apical connector according to claim 14, wherein the fourth frame is provided with a plurality of fourth through holes, the plurality of fourth through holes are spaced apart along the circumferential direction of the first through hole, and the cuff is fixed to the fourth through hole through sutures.

19. The apical connector according to claim 18, wherein the first positioning portion is provided on an inner wall of the fourth frame and is located at an end of the fourth frame facing the first direction.

20. The apical connector according to claim 1, further comprising:
a blocking member comprising;
a blocking base comprising an insertion portion inserted into the second through hole, the second connection assembly being provided outside the insertion portion; and
a plug sleeved on the insertion portion and arranged sequentially with the second connection assembly along an axial direction of the second through hole.

21-25. (canceled)
